(19) United States
(12) Patent Application Publication
(10) Pub. No.: US 2010/0087893 A1
(43) Pub. Date: Apr. 8, 2010

(54) Operant Conditioning-Based Device for Snoring and Obstructive Sleep Apnea and Method of Use

(76) Inventor: Solange Pasquet, Miami, FL (US)

Correspondence Address: ROBERT M. SCHWARTZ, P.A., P.O. BOX 221470, HOLLYWOOD, FL, 33022, US

(21) Appl. No.: 12/573,195

(22) Filed: Oct. 5, 2009

Related U.S. Application Data
(60) Provisional application No. 61/102,424, filed on Oct. 3, 2008.

(51) Int. Cl. A61F 5/56; A61N 1/36

(52) U.S. Cl. 607/48; 128/848

(57) ABSTRACT

The present invention is an apparatus and method for treating, preventing, and ameliorating sleep-related breathing disorders wherein the apparatus includes a central frame positioned between the upper teeth on the hard palate of the user and a movable response lever extending inward from the central frame toward the throat and soft palate of the user.

### INDEX TO RELATED APPLICATIONS

This application claims benefit of U.S. Provisional Patent No. 61/102,424 filed Oct. 3, 2008, the disclosure of which is incorporated herein by reference in its entirety.

### BACKGROUND OF THE INVENTION

Snoring and Obstructive Sleep Apnea (OSA) occur when the soft palate muscles collapse against the posterior pharyngeal wall lining and either partially or completely obstruct the passage of air in the nasopharynx and oropharix thereby crippling or completely impeding airflow.

While many attempts have been made to reduce or eliminate Snoring and OSA, the present invention provides an apparatus and method that is particularly effective since it is intelligent, customized to each consumer, and based on operant conditioning techniques.

### BRIEF SUMMARY OF THE INVENTION

The present invention is an apparatus for treating, preventing, and ameliorating sleep-related breathing disorders. The apparatus includes a central frame positioned between the upper teeth on the hard palate of the user and a movable response lever (or 2) extending inward from the central frame toward the throat and soft palate of the user.

The apparatus of the present invention is preferably removable and mounted on the hard palate. In one embodiment the detachable mounting is a mechanism that secures the apparatus to the upper teeth.

In one embodiment the apparatus has a sensor that may be calibrated such that normal operation does not interfere with normal airflow episodes, such as: breathing, speaking, coughing, laughing, sneezing, yawning, clearing of the throat, swallowing, gargling, spitting, throat scratching or combinations thereof. The sensor may be calibrated based on movement of the response lever. The response lever monitors typical contraction and relaxation patterns of the muscles in the soft palate.

In a preferred embodiment, the response lever is on at least one hinge and interacts with a spring that urges the response lever upward against the soft palate. In an alternative embodiment, the response lever moves by interaction with at least two hinges.

Preferably there is at least one electro-muscular stimulation (EMS) device that is positioned on the soft palate/pressing against the soft palate. The EMS will deliver between about 1 to 5 Volts (v) of electricity when actuated by a microprocessor that controls the apparatus.

In a preferred embodiment there are at least two EMS devices placed on or near the specific muscles responsible for the contraction of the soft palate (namely the tensor palatini and levator palitini muscles, which are located on either side of the throat). Electrical stimulation of these muscles will cause them to contract. The contraction of these muscles produces an elevation of the soft palate. It is the elevation the soft palate that translates to an unobstructed airway thereby providing the desired treatment, prevention, and amelioration of sleep-related breathing disorders.

In a preferred embodiment the EMS devices will contain a gold/gold-based or a copper/copper-based conductor surrounded by a suitable insulator. Suitable insulators are known in the art, however suitable insulators may include silicone rubber and related materials.

The present invention is also a method for treating, preventing, and ameliorating sleep-related breathing disorders comprising the steps of providing an apparatus as described herein and positioning the apparatus on the hard palate. The method of treatment further comprises calibrating the apparatus so as not to interfere with normal airflow episodes. Normal airflow episodes, as used herein, refer to, but are not limited to breathing, speaking, coughing, laughing, sneezing, yawning, clearing of the throat, swallowing, gargling, spitting, throat scratching or combinations thereof.

The calibrating of the apparatus includes storing information related to all patterns of muscle contraction and relaxation of the soft palate in an electronic memory; this includes normal airflow patterns as well as patterns of Snoring and OSA. Muscle contraction and relaxation patterns are monitored using the apparatus. When the patterns of muscle contraction and relaxation detected by the apparatus are congruous with Snoring and OSA patterns stored in electronic memory, instructions are sent to close an electrical circuit within the apparatus and consequently deliver electro-muscular stimulation to the soft-palate muscles.

### BRIEF DESCRIPTION OF THE DRAWINGS

FIG. 1 is a plan view of the device.

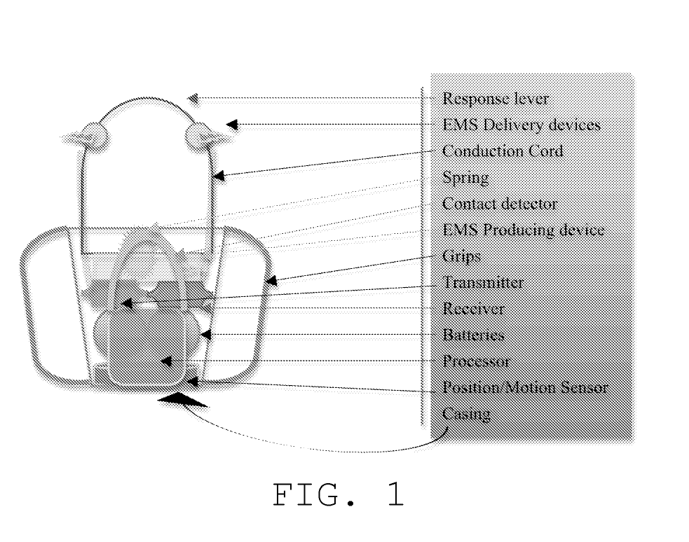

### DETAILED DESCRIPTION OF THE PREFERRED EMBODIMENTS

This device will deliver mild electro-muscular stimulation (EMS) to the pertinent muscles of the soft palate contingent on the response-specific pattern of collapse (or relaxation) of these muscles.

Furthermore, it will be calibrated to each user in order to assure EMS delivery contingent on the specific response topographies targeted (Snoring and OSA responses). Calibration with customized software will ensure that the stimulus is not triggered during normal airflow episodes such as: breathing, speaking, coughing, laughing, sneezing, yawning, clearing of the throat, swallowing, gargling, spitting, throat scratching or any other response topographies.

The device will be constructed to preferably rest on the hard palate (roof of the mouth) and attach to the upper teeth on both sides of the mouth. Furthermore, the device will have an extension (response lever), which will be positioned parallel to the soft palate and will make contact with the soft palate at all times in order to measure patterns of its rise (contraction) and fall (relaxation).

In a preferred embodiment the device will be configured as follows:

### Physical Attributes

### Casing

Envelops all functioning mechanisms in a waterproof (e.g. silicon) medium, including the following elements:
   Processor
      Interprets response lever activity
      Stores/implements instructions for EMS delivery
      Manages information upload/download between input/output devices (transmitter, receiver)
   Batteries
      Provide power for processor, EMS, transmitter and receiver
   EMS-producing device
      Produces electrical stimulation
   Transmitter
      Transmits response lever activity
      Transmits stimulus activity
      Transmits low battery/any malfunctioning signals
   Receiver
      Receives software instructions
   Position/Motion Sensor
      Senses the sleeping position of the consumer (either on his/her back, right side, left side, belly facing right, or belly facing left) since soft palate behavior may differ as a function of sleeping position.
   Response lever mechanism
      Spring attached to bottom-inner portion of response lever gently pushes lever to ceiling of mouth
      Contact detector located at top inner portion of lever detects patterns of muscle contraction/collapse (linked to processor)
         Response lever
Response lever
   Protrusion from casing to back edge of soft palate (adjustable)
EMS delivering devices (2)
   1 positioned at right, 1 at left of response lever
   Targeting left and right tensor palatini and levator palatini muscles (these muscles contract to elevate the soft palate)
   Deliver stimulus to pertinent muscles
Conduction cords
   Link EMS producing device to both EMS delivering devices
   Cord is thread inside response lever tubes

### Grip

Secures device to molars on both sides (adjustable)

### Exterior Components

Transmitter/receiver with built-in auditory alarm
Computer software
   Interprets transmissions
   Defines/categorizes response topographies during calibration (based on user input)

### Calibration Procedure

Device is fitted and adjusted to physically fit consumer

Consumer is asked to engage in the following actions while soft palate activity is recorded and topographies are defined/categorized in terms of patterns of response lever activity:
Breathing through the nose,
Breathing through the mouth,
Speaking,
Coughing,
Laughing,
Sneezing,
Yawning,
Clearing of the throat,
Swallowing,
Gargling liquid
Spitting
Throat scratching

Consumer wears device to sleep while soft palate activity (patterns of muscle contraction and relaxation) is recorded and topographies are defined/further categorized in terms of patterns of response lever activity. During sleep soft palate activities may include the above as well as Snoring, and OSA.

### Program Algorithm for Calibration

For each of the above responses (natural airflow episodes, Snoring, or OSA, repeat the following steps:

| | | | | | | |
|---|---|---|---|---|---|---|
| Consumer | → | Response | → | operator | → | Response lever |
| engages in | | lever | | codes in | | activity is |
| response | | activity is | | response | | ‘paired’ with |
| | | recorded | | topography | | response topography |
| | | | | | | code and stored for |
| | | | | | | later referencing |

### Treatment Implementation Procedure

Once all soft palate topographies/soft palate contraction patterns are defined in terms of response lever activity, software instructions will be uploaded to the device, which will enable it to deliver EMS immediately following
Episodes of snoring
Episodes of OSA

During all other topographies, EMS will not be implemented.

### Program Algorithm for Treatment

For each of the above responses, repeat the following steps:

| | | | | |
|---|---|---|---|---|
| Consumer | → | Program | → | If emitted response |
| engages in | | compares | | matches “snoring” |
| response | | response to | | topography, EMS is |
| | | saved | | delivered |
| | | topographies | | |
| | | | → | If emitted response |
| | | | | matches “OSA” topography, |
| | | | | EMS is delivered |
| | | | → | If it does not match OSA |
| | | | | or snoring topographies, |
| | | | | EMS is not delivered |

Once the stimulus has been delivered, it is expected that the soft palate muscles will contract to clear the airway, thereby preventing further Snoring/OSA. If this does not occur within several seconds, the stimulus will be delivered once again. In the case of OSA, if the soft palate remains collapsed after 2 rounds of stimulus administration, the device will transmit a signal to the outside receiver to initiate the auditory alarm.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

1. An apparatus for treating, preventing and ameliorating sleep-related breathing disorders comprising:
a. a central frame positioned between the upper teeth on the hard palate of a user;
b. a movable response lever extending inward from the central frame toward the soft palate.

2. The apparatus of claim 1 detachably mounted on the hard palate.

3. The apparatus of claim 1 detachably mounted into position through a mechanism that secures the apparatus to the upper teeth.

4. The apparatus of claim 1 having a calibratable sensor that prevents the apparatus from interfering with normal airflow episodes such as breathing, speaking, coughing, laughing, sneezing, yawning, clearing of the throat, swallowing, gargling, spitting, throat scratching or combinations thereof.

5. The apparatus of claim 4 wherein the sensor is calibratable based on movement of the response lever.

6. The apparatus of claim 1 wherein the response lever monitors patterns of contraction and relaxation of muscles in the soft palate.

7. The apparatus of claim 1 having a mechanism for delivering electro-muscular stimulation to the soft palate.

8. The apparatus of claim 1 wherein a mechanism is positioned on the response lever for delivering electro-muscular stimulation to the soft palate.

9. An oral apparatus comprising:
a. a central frame having a casing, wherein said casing houses a battery, a receiver, a transmitter, a microprocessor and a sensor; and
b. a movable response lever that electronically interacts with the sensor.

10. (canceled)

11. (canceled)

12. (canceled)

13. (canceled)

14. (canceled)

15. (canceled)

16. A method for treating, preventing and ameliorating sleep-related breathing disorders comprising:
a. providing an apparatus according to claim 1;
b. positioning the apparatus on the hard palate.

17. The method of claim 16 further including calibrating the apparatus so as to not interfere with normal airflow episodes.

18. The method of claim 17 wherein the normal airflow episodes are breathing, speaking, coughing, laughing, sneezing, yawning, clearing of the throat, swallowing, gargling, spitting, throat scratching or combinations thereof.

19. The method of claim 17 including the step of storing information in an electronic memory relating to all patters of muscle contraction and relaxation of the soft palate including normal airflow episodes as well as patterns of Snoring and OSA.

20. The method of claim 19 wherein the patterns of muscle contraction/relaxation are compared with patterns of muscle contraction/relaxation of all airflow episodes previously stored in the electronic memory.

21. The method of claim 20 wherein the apparatus is signaled when the patterns of muscle contraction/relaxation detected are congruous with patterns of Snoring or OSA episodes stored in the electronic memory, and instructions are sent to activate the transmission of electro-muscular stimulation to the soft palate muscles.
